(19) United States
(12) Patent Application Publication
(10) Pub. No.: US 2026/0355077 A1
(43) Pub. Date: Nov. 12, 2026

(54) WATER-IN-OIL EMULSION COSMETIC

(71) Applicant: SHISEIDO COMPANY, LTD., Chuo-ku, Tokyo (JP)

(72) Inventors: Megumi SAITO, Tokyo (JP); Kosuke ARIMOTO, Tokyo (JP)

(73) Assignee: SHISEIDO COMPANY, LTD., Chuo-ku, Tokyo (JP)

(21) Appl. No.: 19/125,903

(22) PCT Filed: Nov. 16, 2023
(86) PCT No.: PCT/JP2023/041183

§ 371 Date: Apr. 30, 2025

(30) Foreign Application Priority Data
Nov. 30, 2022 (JP) 2022-191222

(51) Int. Cl. A61K 8/19; A61K 8/06; A61K 8/25; A61K 8/44; A61Q 17/04

(52) U.S. Cl. CPC A61K 8/19; A61K 8/064; A61K 8/25; A61K 8/44; A61Q 17/04; A61K 2800/612; A61K 2800/622; A61K 2800/624; A61K 2800/651

(57) ABSTRACT

An objective of the invention is to provide a water-in-oil emulsion cosmetic that, despite containing tranexamic acid, has little stickiness, has excellent properties in texture such as a feeling of fitting and smoothness, and also has excellent glossiness and uniformity of finish. The water-in-oil emulsion cosmetic according to the present invention is characterized by comprising (A) tranexamic acid, (B) 3% to 9% by mass of a hydrophobized mica and (C) an organically modified clay mineral.

### TECHNICAL FIELD

The present invention relates to a water-in-oil emulsion cosmetic that, despite containing tranexamic acid, which tends to cause stickiness, has excellent properties in texture and a good finish.

### BACKGROUND ART

Stimulation by UV rays from sunlight induces the generation of melanin pigments in skin, and when these become abnormally deposited in the skin, they cause spots and freckles. In order to prevent such spots and freckles due to sunburn, tranexamic acid has conventionally been widely blended as a whitener.

However, tranexamic acid had the problem that, when blended into cosmetics, the smoothness and feeling of fitting (appropriate spreading performance) when applied were poor, and the glossiness and uniformity of the finish were inferior. Furthermore, these problems tended to be particularly prominent in water-in-oil emulsion cosmetics containing oils in abundance. That is, while water-in-oil emulsion type cosmetics have the advantages of excellent smoothness, moistness, makeup retention, etc. compared with aqueous or oil-in-water emulsion type cosmetics, they have a poor feeling of freshness and often have a sticky or oily texture. Therefore, when tranexamic acid is blended into a water-in-oil emulsion cosmetic, the stickiness becomes even more prominent and the texture tends to become significantly worse. For this reason, cosmetics in which tranexamic acid is blended are generally of types with relatively less oils, in which the aqueous components constitute the continuous phase. For example, they are often aqueous cosmetics as disclosed in Patent Document 1, etc., or oil-in-water emulsion cosmetics as described in Patent Documents 2 and 3, etc.

Meanwhile, water-in-oil emulsion cosmetics in which tranexamic acid is blended, though few in number, have been proposed. For example, Patent Document 4 discloses a water-in-oil emulsion composition containing tranexamic acid, a prescribed polyoxyalkylene-modified silicone and a prescribed linear silicone. This composition is described as not feeling sticky and providing a watery texture.

However, the suppression of stickiness still cannot be considered to attain an adequate level, and further improvements are sought in terms of the properties in texture and the finish.

### RELATED ART

### Patent Documents

Patent Document 1: JP 2021-161077 A
Patent Document 2: JP 2019-178125 A
Patent Document 3: JP 2022-063613 A
Patent Document 4: JP 2020-083763 A

### SUMMARY OF THE INVENTION

### Problem to be Solved by the Invention

The present invention was made in consideration of the above-mentioned circumstances, and an objective thereof is to provide a water-in-oil emulsion cosmetic that, despite containing tranexamic acid, has little stickiness and has excellent properties in texture and finish.

### Means for Solving the Problem

The present inventors carried out diligent investigations towards solving the above-mentioned problem, as a result of which they discovered that the above-mentioned problem can be solved by blending, in a water-in-oil emulsion cosmetic containing tranexamic acid, prescribed amounts of a hydrophobized mica and an organically modified clay mineral, thereby completing the present invention.

That is, the gist of the present invention is a water-in-oil emulsion cosmetic comprising:
(A) tranexamic acid;
(B) 3% to 9% by mass of a hydrophobized mica; and
(C) an organically modified clay mineral.

### Effects of the Invention

The water-in-oil emulsion cosmetic according to the present invention, by having the features described above, can provide a water-in-oil emulsion cosmetic that can suppress stickiness caused by blending tranexamic acid, that has excellent properties in texture such as a feeling of fitting and smoothness, and that achieves adequate glossiness and a uniform finish.

Additionally, in addition to having skin whitening effects and skin irritation improvement effects due to the blending of tranexamic acid, it also has effects of covering (concealing) unevenness of color, texture irregularities, etc. of the skin due to the blending of a hydrophobized mica.

### MODES FOR CARRYING OUT THE INVENTION

The water-in-oil emulsion cosmetic of the present invention is characterized by comprising (A) tranexamic acid, (B) a hydrophobized mica and (C) an organically modified clay mineral. Hereinafter, the respective components constituting the water-in-oil emulsion cosmetic of the present invention will be explained in detail.

### <(A) Tranexamic Acid>

The (A) tranexamic acid in the present invention includes tranexamic acid (trans-4-aminomethylcyclohexane-1-carboxylic acid) and derivatives or salts thereof.

Tranexamic acid derivatives include, for example, esters, amides, etc. Tranexamic acid esters include tranexamic acid lauryl ester, tranexamic acid hexadecyl ester, tranexamic acid cetyl ester, etc., and tranexamic acid amides include tranexamic acid methyl amide, etc.

Salts of tranexamic acid or derivatives thereof include alkali metal salts such as sodium salts and potassium salts; alkaline earth metal salts such as calcium salts and magnesium salts; zinc salts; iron salts; ammonium salts; salts with basic amino acids such as arginine, histidine, lysine and ornithine; and salts with amines such as monoethanolamine, diethanolamine and triethanolamine; etc.

The blended amount of the (A) tranexamic acid in the water-in-oil emulsion cosmetic of the present invention is preferably 0.1% by mass or more, more preferably 2% by mass or more, and preferably 5% by mass or less, more preferably 3% by mass or less with respect to the overall amount of the cosmetic. Thus, the blended amount range may be 0.1% by mass to 5% by mass, 2% by mass to 3% by mass, etc. Within these ranges, the skin whitening effects and skin irritation improvement effects of tranexamic acid are more easily obtained, and decreases in the properties in texture due to stickiness do no tend to occur.

### <(B) Hydrophobized Mica>

The mica used in the (B) hydrophobized mica may be either natural or synthetic, and may include, for example, muscovite, biotite, phlogopite, synthetic mica, etc.

The hydrophobization treatment of the mica surface may be a dialkyl dimethyl ammonium salt treatment by distearyldimonium chloride, etc.; a silicone treatment with methyl polysiloxane, methylhydrogen polysiloxane, triethoxysilylethyl polydimethylsiloxyethyl dimethicone, etc.; an amino-modified silicone treatment with amodimethicone, aminopropyl triethoxysilane, etc.; a fatty acid treatment with stearic acid, etc.; a metal soap treatment with magnesium stearate, aluminum stearate, magnesium myristate, etc.; a dextrin fatty acid treatment with dextrin palmitate, etc.; a silane coupling treatment with octyl triethoxysilane, etc.; a fluorine compound treatment with perfluorohexylethyl triethoxysilane, etc.; an amino acid treatment with N-acylglutamic acid; etc., among which one or more types may be used. In particular, one or more types selected from the group consisting of distearyldimonium chloride, amodimethicone, magnesium stearate and triethoxysilylethyl polydimethylsiloxyethyl dimethicone are preferable. The treatment method is not particularly limited, and the treatment may be by a method that is known for each treatment agent.

The average primary particle size of the (B) hydrophobized mica is, normally, preferably 30 μm or smaller, more preferably 20 μm or smaller, and preferably 1 μm or larger, more preferably 5 μm or larger. The average primary particle size is measured by a conventional method such as the number-average size by image analysis of an electron microscope photograph.

The blended amount of the (B) hydrophobized mica in the water-in-oil emulsion cosmetic of the present invention is 3% by mass or more, more preferably 5% by mass or more, and 9% by mass or less, more preferably 7% by mass or less with respect to the overall amount of the cosmetic. Thus, the blended amount range may be 3% by mass to 9% by mass, 5% by mass to 7% by mass, etc. Within these ranges, in addition to being able to adequately suppress stickiness caused by blending tranexamic acid, the effects of covering unevenness of color, texture irregularities, etc. of the skin can also be adequately obtained.

### <(C) Organically Modified Clay Mineral>

The (C) organically modified clay mineral is a type of colloidal hydrated aluminum silicate having a three-layered structure, modified by a quaternary ammonium salt type cationic surfactant, represented by the following general formula (I):

(X,Y)₂₋₃(Si,Al)₄O₁₀(OH)₂Z_{1/3}·nH₂O (I)

where X═Al, Fe(III), Mn(III) or Cr(III); Y═Mg, Fe(II), Ni, Zn or Li; and Z═K, Na or Ca.

Specific examples of the (C) organically modified clay mineral include dimethyl distearyl ammonium hectorite (disteardimonium hectorite), dimethyl alkyl ammonium hectorite, benzyl dimethyl stearyl ammonium hectorite, distearyl dimethyl ammonium chloride-treated aluminum-magnesium silicate, etc. Among the above, disteardimonium hectorite is particularly preferred.

Commercial products include Bentone 27 (benzyl dimethyl stearyl ammonium chloride-treated hectorite, manufactured by Elementis Japan KK) and Bentone 38 (distearyl dimethyl ammonium chloride-treated hectorite, manufactured by Elementis Japan KK).

The blended amount of the (C) organically modified clay mineral in the water-in-oil emulsion cosmetic of the present invention is 0.1% by mass or more, more preferably 0.5% by mass or more, and 5% by mass or less, more preferably 2% by mass or less with respect to the overall amount of the cosmetic. Thus, the blended amount range may be 0.1% by mass to 5% by mass, 0.5% by mass to 2% by mass, etc. Within this range, stickiness caused by tranexamic acid may be suppressed, and excellent smoothness and a feeling of fitting can be realized.

### <Optional Blended Components>

In addition to components (A) to (C) above, components that are normally used in cosmetics, for example, oil-based components, UV protectants, surfactants, polyhydric alcohols, powder components, etc. can be blended, as appropriate and as needed, into the water-in-oil emulsion cosmetic of the present invention.

The oil-based components include, for example, oils/fats such as camellia oil, macadamia nut oil, olive oil, castor oil, safflower oil, soybean oil, tea seed oil, cacao fat, coconut oil, hydrogenated coconut oil, palm oil, Japan wax, hydrogenated castor oil, beeswax, candelilla wax, carnauba wax, lanolin, liquid lanolin, jojoba wax, hard lanolin, polyoxyethylene lanolin alcohol ether and polyoxyethylene cholesteryl ether; hydrocarbon oils such as fluid paraffin, ozokerite, squalane, paraffin, ceresin, vaseline and microcrystalline wax; synthetic oil-based components such as isopropyl myristate, octyldodecyl myristate, isopropyl palmitate, cholesteryl 12-hydroxystearate, ethylene glycol di-2-ethylhexylate, dipentaerythritol fatty acid ester, pentaerythritol tetra-2-ethylhexylate, glycerol tri-2-ethylhexylate, trimethylolpropane triisostearate, cetyl-2-ethylhexanoate and castor oil fatty acid methyl ester; linear polysiloxanes such as dimethyl polysiloxane, methylphenyl polysiloxane and methylhydrogen polysiloxane; and cyclic polysiloxanes such as decamethyl polysiloxane, dodecamethyl polysiloxane and tetramethyl tetrahydrogen polysiloxane; etc.

As the UV protectants, UV absorbing agents and UV scattering agents that are normally blended in cosmetics can be used. UV absorbing agents include ethylhexyl methoxycinnamate, octocrylene, dimethicodiethyl benzalmalonate, polysilicone-15, t-butyl methoxy dibenzoylmethane, ethylhexyl triazone, diethylamino hydroxybenzoyl hexyl benzoate, bis-ethylhexyloxyphenol methoxyphenyl triazine, oxybenzone-3, methylene bis-benzotriazolyl tetramethylbutylphenol, phenylbenzimidazole sulfonic acid, homosalate, ethylhexyl salicylate, etc. Additionally, UV scattering agents include fine-particle metal oxides, for example, zinc oxide, titanium oxide, iron oxide, cerium oxide, tungsten oxide or the above that have been subjected to hydrophobizing surface treatments, etc.

The surfactants are not particularly limited as long as they are surfactants with an HLB of 8 or lower in order to realize a water-in-oil emulsion form, and silicone-based surfactants are particularly preferable. Such silicone-based surfactants include, for example, polyoxyalkylene-modified silicones, polyoxyalkylene/alkyl-comodified silicones, polyglycerin-modified silicones and/or polyglycerin/alkyl-comodified silicones.

The polyhydric alcohols include, for example, ethylene glycol, propylene glycol, dipropylene glycol, 1,3-butylene glycol, glycerin, erythritol, trimethylolpropane, pentaerythritol, sorbitol, maltitol, diglycerin, polyethylene glycol, etc.

The powder components include, for example, inorganic powders such as talc, kaolin, magnesium carbonate, calcium carbonate, aluminum silicate, silica, zeolite, barium sulfate, calcined calcium sulfate (calcined plaster), calcium phosphate, fluorapatite, hydroxyapatite, ceramic powder, metal soaps (zinc myristate, calcium palmitate, aluminum stearate, etc.) and boron nitride; organic powders such as polyamide resin powder (nylon powder), polyethylene powder, methyl polymethacrylate powder, polystyrene powder, styrene and acrylic acid copolymer resin powder, polymethyl silsesquioxane powder and cellulose powder; inorganic pigments such as titanium dioxide, iron oxide (Bengal red), iron titanate, yellow iron oxide, black iron oxide, carbon black, low-order titanium oxide, mango violet, cobalt violet, chromium oxide, ultramarine blue and Prussian blue; pearlescent pigments such as titanium dioxide-coated mica, titanium dioxide-coated bismuth oxychloride, colored titanium dioxide-coated mica, bismuth oxychloride and argentine; metallic powder pigments such as aluminum powder; organic pigments such as zirconium lakes, barium lakes and aluminum lakes of Red No. 201, Red No. 202, Orange No. 203, Yellow No. 205, Yellow No. 401, Blue No. 404, Red No. 3, Yellow No. 4, Green No. 3, Blue No. 1, etc.; natural pigments such as chlorophyll and β-carotene; etc.

The water-in-oil emulsion cosmetic of the present invention can be prepared in a low-viscosity product form such as a milky liquid or a lotion. For example, it should preferably be provided as a skin-care milky liquid provided with whitening effects, skin quality improvement effects, skin correction effects, sunscreen effects, etc.

### EXAMPLES

Though the present invention will be explained in further detail by providing examples below, the present invention is not limited in any way by these examples. Where not specially noted, the blended amounts are indicated in percentage by mass with respect to the water-in-oil emulsion cosmetic. Before specifically explaining the respective examples, the evaluation methods that were employed will be explained.

### <Texture>

Prepared cosmetics were actually used by expert panelists and evaluated in five levels regarding the (1) lack of stickiness and the (2) smoothness when applied to skin.
A+: Extremely good
A: Good
B: Slightly poor
C: Poor
D: Very poor

Prepared cosmetics were actually used by expert panelists and evaluated in three levels regarding the (3) feeling of fitting when applied to skin.
A+: Extremely good
A: Good
B: Poor

### <Finish>

Prepared cosmetics were actually used by expert panelists and evaluated in five levels regarding the (4) glossiness and the (5) uniformity after being applied to skin.
A+: Extremely good
A: Good
B: Slightly poor
C: Poor
D: Very poor

### Examples 1 to 4 and Comparative Examples 1 to 5

The water-in-oil emulsion cosmetics having the compositions described in Table 1 below were prepared and evaluated regarding properties in texture and finish.

TABLE 1

| | Ref. | Comp. | Comp. | Comp. | Comp. | Comp. | Ex. | Ex. | Ex. | Ex. |
|---|---|---|---|---|---|---|---|---|---|---|
| | Ex. | Ex. 1 | Ex. 3 | Ex. 3 | Ex. 4 | Ex. 5 | 1 | 2 | 3 | 4 |
| PEG-9 polydimethyl siloxyethyl | 2 | 2 | 2 | 2 | 2 | 2 | 2 | 2 | 2 | 2 |
| dimethicone | | | | | | | | | | |
| bis-Butyldimethicone | 2 | 2 | 2 | 2 | 2 | 2 | 2 | 2 | 2 | 2 |
| polyglyceryl-3 | | | | | | | | | | |
| PEG-10 dimethicone | 2 | 2 | 2 | 2 | 2 | 2 | 2 | 2 | 2 | 2 |
| Dimethicone (1.5 cs) | 6 | 6 | 6 | 6 | 6 | 6 | 6 | 6 | 6 | 6 |
| Dimethicone (6 cs) | 10 | 10 | 10 | 10 | 10 | 10 | 10 | 10 | 10 | 10 |
| Diphenylsiloxyphenyl | 8 | 8 | 8 | 8 | 8 | 8 | 8 | 8 | 8 | 8 |
| trimethicone | | | | | | | | | | |
| Fragrance | s.a. | s.a. | s.a. | s.a. | s.a. | s.a. | s.a. | s.a. | s.a. | s.a. |
| Ethylhexyl methoxycinnamate | 7 | 7 | 7 | 7 | 7 | 7 | 7 | 7 | 7 | 7 |
| Diethylamino hydroxybenzoyl | 0.5 | 0.5 | 0.5 | 0.5 | 0.5 | 0.5 | 0.5 | 0.5 | 0.5 | 0.5 |
| hexyl benzoate | | | | | | | | | | |
| Disteardimonium hectorite | 0.5 | 0.5 | 0.5 | 0.5 | 0.5 | — | 0.5 | 0.5 | 0.5 | 0.5 |
| Fine-particle titanium oxide | 7.5 | 7.5 | 7.5 | 7.5 | 7.5 | 7.5 | 7.5 | 7.5 | 7.5 | 7.5 |
| Hydrophobized mica¹ | 5 | — | — | — | — | 5 | 5 | — | — | — |
| Hydrophobized mica² | — | — | — | — | — | — | — | 5 | — | — |
| Hydrophobized mica³ | — | — | — | — | — | — | — | — | 5 | — |
| Hydrophobized mica⁴ | — | — | — | — | — | — | — | — | — | 5 |
| Synthetic phlogopite | — | — | 5 | — | — | — | — | — | — | — |
| Hydrophobized talc⁵ | — | — | — | 5 | — | — | — | — | — | — |
| Hydrophobized silica⁶ | — | — | — | — | 5 | — | — | — | — | — |
| Silicone-treated red iron oxide | 0.5 | 0.5 | 0.5 | 0.5 | 0.5 | 0.5 | 0.5 | 0.5 | 0.5 | 0.5 |
| Silicone-treated yellow iron oxide | 1 | 1 | 1 | 1 | 1 | 1 | 1 | 1 | 1 | 1 |
| Silicone-treated black iron oxide | 0.01 | 0.01 | 0.01 | 0.01 | 0.01 | 0.01 | 0.01 | 0.01 | 0.01 | 0.01 |
| Silicone-treated pigment-grade | 6.5 | 6.5 | 6.5 | 6.5 | 6.5 | 6.5 | 6.5 | 6.5 | 6.5 | 6.5 |
| titanium oxide | | | | | | | | | | |
| Purified water | bal | bal | bal | bal | bal | bal | bal | bal | bal | bal |
| Tranexamic acid | — | 2 | 2 | 2 | 2 | 2 | 2 | 2 | 2 | 2 |
| Glycerin | 5 | 5 | 5 | 5 | 5 | 5 | 5 | 5 | 5 | 5 |
| Dipropylene glycol | 8 | 8 | 8 | 8 | 8 | 8 | 8 | 8 | 8 | 8 |
| Citric acid | 0.1 | 0.1 | 0.1 | 0.1 | 0.1 | 0.1 | 0.1 | 0.1 | 0.1 | 0.1 |
| Sodium citrate | 0.1 | 0.1 | 0.1 | 0.1 | 0.1 | 0.1 | 0.1 | 0.1 | 0.1 | 0.1 |
| EDTA-3Na•H2O | 0.2 | 0.2 | 0.2 | 0.2 | 0.2 | 0.2 | 0.2 | 0.2 | 0.2 | 0.2 |
| Preservative | s.a. | s.a. | s.a. | s.a. | s.a. | s.a. | s.a. | s.a. | s.a. | s.a. |
| Total | 100 | 100 | 100 | 100 | 100 | 100 | 100 | 100 | 100 | 100 |
| Lack of stickiness | A+ | D | C | B | D | B | A | A | A | A |
| Feeling of fitting | A | A | A | A | A | B | A+ | A | A+ | A+ |
| Smoothness | A | B | C | B | D | C | A+ | A+ | A+ | A+ |
| Glossiness | A+ | A | A | B | D | A+ | A+ | A | A+ | A+ |
| Uniformity | A | C | A+ | A+ | C | A+ | A+ | A+ | A+ | A+ |

¹Hydrophobized mica obtained by treating the surface of synthetic phlogopite with distearyldimonium chloride
²Hydrophobized mica obtained by treating the surface of synthetic phlogopite with amodimethicone
³Hydophobized mica obtained by treating the surface of synthetic phlogopite with magnesium stearate
⁴Hydrophobized mica obtained by treating the surface of synthetic phlogopite with triethoxysilylethyl polydimethylsiloxyethyl dimethicone
⁵Hydrophobized talc obtained by treating the surface of talc with dimethicone
⁶Hydrophobized silica obtained by treating the surface of silica with dimethicone

As indicated in Table 1 above, in the case in which (A) tranexamic acid was not blended, no particular problems were observed regarding the properties in texture and the finish (Reference Example). However, when (A) tranexamic acid was blended, significant stickiness occurred, the smoothness of application was lost, and the uniformity of finish was also not adequate (Comparative Example 1). Additionally, the reductions in the properties in texture and the finish due to blending (A) tranexamic acid were not improved even when blending untreated mica (synthetic phlogopite), hydrophobized talc or hydrophobied silica (Comparative Examples 2 to 4). Meanwhile, when a (B) hydrophobized mica was blended, the lack of stickiness was largely improved, and also, excellent results were exhibited in all other evaluation categories (Examples 1 to 4).

Additionally, when a (C) organically modified clay mineral was not blended, it was observed that the properties in texture such as lack of stickiness, the feeling of fitting and the smoothness were inferior (Comparative Example 5).

### Examples 5 to 10 and Comparative Examples 6 to 8

The water-in-oil emulsion cosmetics having the compositions described in Table 2 below were prepared and evaluated regarding properties in texture and finish.

TABLE 2

| | Comp. | Comp. | Ex. | Ex. | Ex. | Ex. | Ex. | Ex. | Comp. |
|---|---|---|---|---|---|---|---|---|---|
| | Ex. 6 | Ex. 7 | 5 | 6 | 7 | 8 | 9 | 10 | Ex. 8 |
| PEG-9 polydimethyl siloxyethyl | 2 | 2 | 2 | 2 | 2 | 2 | 2 | 2 | 2 |
| dimethicone | | | | | | | | | |
| bis-Butyldimethicone polyglyceryl-3 | 2 | 2 | 2 | 2 | 2 | 2 | 2 | 2 | 2 |
| PEG-10 dimethicone | 2 | 2 | 2 | 2 | 2 | 2 | 2 | 2 | 2 |
| Dimethicone (1.5 cs) | 6 | 6 | 6 | 6 | 6 | 6 | 6 | 6 | 6 |
| Dimethicone (6 cs) | 10 | 10 | 10 | 10 | 10 | 10 | 10 | 10 | 10 |
| Diphenylsiloxyphenyl trimethicone | 8 | 8 | 8 | 8 | 8 | 8 | 8 | 8 | 8 |
| Fragrance | s.a. | s.a. | s.a. | s.a. | s.a. | s.a. | s.a. | s.a. | s.a. |
| Ethylhexyl methoxycinnamate | 7 | 7 | 7 | 7 | 7 | 7 | 7 | 7 | 7 |
| Diethylamino hydroxybenzoyl hexyl | 0.5 | 0.5 | 0.5 | 0.5 | 0.5 | 0.5 | 0.5 | 0.5 | 0.5 |
| benzoate | | | | | | | | | |
| Disteardimonium hectorite | 0.5 | 0.5 | 0.5 | 0.5 | 0.5 | 0.5 | 0.5 | 0.5 | 0.5 |
| Fine-particle titanium oxide | 7.5 | 7.5 | 7.5 | 7.5 | 7.5 | 7.5 | 7.5 | 7.5 | 7.5 |
| Hydrophobized mica¹ | 1 | 2 | 3 | 4 | 6 | 7 | 8 | 9 | 10 |
| Silicone-treated red iron oxide | 0.5 | 0.5 | 0.5 | 0.5 | 0.5 | 0.5 | 0.5 | 0.5 | 0.5 |
| Silicone-treated yellow iron oxide | 1 | 1 | 1 | 1 | 1 | 1 | 1 | 1 | 1 |
| Silicone-treated black iron oxide | 0.01 | 0.01 | 0.01 | 0.01 | 0.01 | 0.01 | 0.01 | 0.01 | 0.01 |
| Silicone-treated pigment-grade titanium | 6.5 | 6.5 | 6.5 | 6.5 | 6.5 | 6.5 | 6.5 | 6.5 | 6.5 |
| oxide | | | | | | | | | |
| Purified water | bal | bal | bal | bal | bal | bal | bal | bal | bal |
| Tranexamic acid | 2 | 2 | 2 | 2 | 2 | 2 | 2 | 2 | 2 |
| Glycerin | 5 | 5 | 5 | 5 | 5 | 5 | 5 | 5 | 5 |
| Dipropylene glycol | 8 | 8 | 8 | 8 | 8 | 8 | 8 | 8 | 8 |
| Citric acid | 0.1 | 0.1 | 0.1 | 0.1 | 0.1 | 0.1 | 0.1 | 0.1 | 0.1 |
| Sodium citrate | 0.1 | 0.1 | 0.1 | 0.1 | 0.1 | 0.1 | 0.1 | 0.1 | 0.1 |
| EDTA-3Na•H2O | 0.2 | 0.2 | 0.2 | 0.2 | 0.2 | 0.2 | 0.2 | 0.2 | 0.2 |
| Preservative | s.a. | s.a. | s.a. | s.a. | s.a. | s.a. | s.a. | s.a. | s.a. |
| Total | 100 | 100 | 100 | 100 | 100 | 100 | 100 | 100 | 100 |
| Lack of stickiness | B | B | A | A | A | A | A | A | B |
| Feeling of fitting | A | A+ | A+ | A+ | A+ | A+ | A+ | A+ | A |
| Smoothness | A | A | A+ | A+ | A+ | A+ | A+ | A+ | A |
| Glossiness | A | A | A+ | A+ | A+ | A+ | A+ | A+ | A+ |
| Uniformity | A+ | A+ | A+ | A+ | A+ | A+ | A+ | A+ | A+ |

As indicated in Table 2 above, the stickiness due to blending (A) tranexamic acid was not able to be adequately suppressed when the blended amount of the (B) hydrophobized mica was less than 3% by mass (Comparative Examples 6 and 7). Additionally, it was also observed that the stickiness was not able to be suppressed when the blended amount of the (B) hydrophobized mica exceeded 9% by mass (Comparative Example 8).

Meanwhile, even when the (B) hydrophobized mica was blended to be within the range 3% to 9% by mass, excellent effects were exhibited in all of the evaluation categories (Examples 5 to 10).

1. A water-in-oil emulsion cosmetic comprising:
(A) tranexamic acid;
(B) 3% to 9% by mass of a hydrophobized mica; and
(C) an organically modified clay mineral.

2. The water-in-oil emulsion cosmetic according to claim 1, wherein a surface treatment agent for the (B) hydrophobized mica is of one or more types selected from the group consisting of distearyldimonium chloride, amodimethicone, magnesium stearate and triethoxysilylethyl polydimethylsiloxyethyl dimethicone.

3. The water-in-oil emulsion cosmetic according to claim 1, wherein the (C) organically modified clay mineral is disteardimonium hectorite.

4. The water-in-oil emulsion cosmetic according to claim 1 further comprising a UV protectant.
